(19) United States
(12) Patent Application Publication
(10) Pub. No.: US 2026/0295214 A1
(43) Pub. Date: Oct. 1, 2026

(54) LOW TRAUMA URINARY CATHETER EYELETS AND METHODS FOR MAKING

(71) Applicant: Hollister Incorporated, Libertyville, IL (US)

(72) Inventor: Damien Biggins, Ballina (IE)

(21) Appl. No.: 19/479,962

(22) PCT Filed: Apr. 30, 2024
(86) PCT No.: PCT/US2024/027042

§ 371 Date: Oct. 29, 2025

Related U.S. Application Data
(60) Provisional application No. 63/463,262, filed on May 1, 2023.

(51) Int. Cl. A61M 25/00

(52) U.S. Cl. CPC A61M 25/007; A61M 25/0017

(57) ABSTRACT

Catheters that include at least one eyelet shaped to decrease urethral trauma on the proximal insertion end portion of the catheter.

### CROSS-REFERENCE TO RELATED APPLICATION

This application claims the benefit of U.S. Provisional Application Ser. No. 63/463,262, filed May 1, 2023, the disclosure of which is hereby incorporated by reference in its entirety.

### FIELD OF THE DISCLOSURE

This disclosure relates generally to urinary catheters. More particularly, this disclosure relates to intermittent urinary catheters with eyelets shaped to reduce the chance of urethral discomfort during catheterization.

### BACKGROUND

Intermittent urinary catheters include a catheter shaft having a proximal insertion end portion that is inserted through the urethra and into the bladder. Once in the bladder, the urine enters the catheter through one or more openings, oftentimes referred to as eyelets. The urine enters into the opening(s) and drains through a drainage lumen of the catheter and out of a distal drainage opening in the distal end portion of the catheter shaft.

FIGS. 1 and 2 illustrate a prior art example of an intermittent urinary catheter. Catheter C includes an elongated catheter shaft S having a proximal insertion end portion P and a distal end portion D. Proximal insertion end portion P includes a terminal proximal end 10 that is suitable for insertion into the urethra. The proximal insertion end portion includes eyelets E1 and E2, shaped in slots or oval openings, for receiving urine therethrough and into an internal conduit or lumen of catheter shaft S. Distal end portion D may include a distal opening that is in fluid communication with a drainage member F, such as a funnel, for fluidly connecting catheter C to a collection container, such as a collection bag, or for directing urine to a waste receptacle, such as a toilet.

The catheter includes longitudinal axis A. Although the catheter is an elongated cylinder, there are a number of surfaces that can be defined in relation to the placement of the catheter eyelets. As shown in FIGS. 1 and 2, there is top surface 13, which is the surface of the first eyelet E1. There is also a second surface, or bottom surface 15, which is the surface of the second eyelet E2.

There are two opposing surfaces, 17 and 19, respectively the left and right surfaces, that are on a perpendicular plane about the axis A from the top and bottom surfaces 13, 15 of the catheter.

Repeat users of intermittent catheters may experience urethral trauma, specifically to the urethral lining, due to repeat insertion and removal of intermittent urinary catheters.

The prior art catheter utilizes two slot shaped eyelets for draining during use. Although this draining may be effective, there is a need for eyelets that increase comfort during insertion and withdrawal of the catheter and reduce the chance of urethral discomfort during catheterization. Catheter eyelets of the current disclosure are shaped to minimize urethral discomfort during catheterization.

### SUMMARY

In one aspect, an intermittent urinary catheter includes a catheter shaft having a proximal insertion end portion for advancement through the urethra into a bladder and a distal drainage end portion having a drainage opening. The catheter shaft has a drainage lumen in communication with the drainage opening. The catheter includes at least one eyelet on the catheter shaft at the proximal insertion end portion. The eyelet has the shape of a diamond.

In another aspect, an intermittent urinary catheter includes a catheter shaft having a proximal insertion end portion for advancement through the urethra into a bladder and a distal drainage end portion having a drainage opening. The catheter shaft has a drainage lumen in communication with the drainage opening. The catheter includes at least one eyelet on the catheter shaft at the proximal insertion end portion. The eyelet includes an outer shape divided into a number of adjoining portions.

In yet another aspect, an intermittent urinary catheter includes a catheter shaft having a proximal insertion end portion for advancement through the urethra into a bladder and a distal drainage end portion having a drainage opening. The catheter shaft has a drainage lumen in communication with the drainage opening. The catheter includes at least one eyelet on the catheter shaft at the proximal insertion end portion. The at least one eyelet includes a middle open hole portion and at least one outer recessed groove portion.

Additionally, in another aspect a method of forming a catheter eyelet on a surface of a catheter includes applying a first die to the surface of the catheter to form an indented catheter surface and applying a second die to the idented surface of the catheter to form an eyelet opening on the surface of the catheter. The resulting eyelet includes an open middle portion and at least one outer recessed portion.

### BRIEF DESCRIPTION OF THE DRAWINGS

FIG. 1 is a perspective view of a prior art urinary catheter;

FIG. 2 is a side view of the proximal insertion end of the prior art urinary catheter of FIG. 1;

FIG. 3 is an enlarged front view of a portion of the proximal insertion end of a urinary catheter with an eyelet of an exemplary embodiment of the current disclosure;

FIG. 4 is an enlarged perspective view of a portion of the proximal insertion end of the urinary catheter of FIG. 3;

FIG. 5 is an enlarged front view of a portion of the proximal insertion end of a urinary catheter with an eyelet of an exemplary embodiment of the current disclosure;

FIG. 6 is an enlarged perspective view of a portion of the proximal insertion end of the urinary catheter of FIG. 5;

FIG. 7 is an enlarged front view of a portion of the proximal insertion end of a urinary catheter with an eyelet of an exemplary embodiment of the current disclosure;

FIG. 8 is an enlarged perspective view of a portion of the proximal insertion end of the urinary catheter of FIG. 7;

FIGS. 9A, 9B and 9C are an enlarged cross-sectional view of a portion of the proximal insertion end of the urinary catheter of FIGS. 7 and 8;

FIG. 10 is an enlarged front view of a portion of the proximal insertion end of a urinary catheter with an eyelet of an exemplary embodiment of the current disclosure;

FIG. 11 is an enlarged perspective view of a portion of the proximal insertion end of the urinary catheter of FIG. 10;

FIGS. 12A, 12B and 12C are an enlarged cross-sectional view of a portion of the proximal insertion end of the urinary catheter of FIGS. 7 and 8;

FIG. 13 is a perspective bottom view of a die for forming a portion of an eyelet of an exemplary embodiment of the current disclosure;

FIG. 14 is a side view of the die of FIG. 13.

FIG. 15 is a perspective view of a compressed catheter wall after applying the die of FIGS. 13 and 14.

FIG. 16 is a perspective view of a die for forming a portion of an eyelet of an exemplary embodiment of the current disclosure;

FIG. 17 is a perspective view of a compressed catheter wall after applying the die of FIG. 16.

FIG. 18 is a perspective bottom view of a die for forming a portion of an eyelet of an exemplary embodiment of the current disclosure;

FIG. 19 is a side view of the die of FIG. 18.

FIG. 20 is a perspective view of a compressed catheter wall after applying the die of FIGS. 18 and 19.

FIG. 21 is a perspective view of a die for forming a portion of an eyelet of an exemplary embodiment of the current disclosure; and

FIG. 22 is a perspective view of a compressed catheter wall after applying the die of FIG. 21.

### DETAILED DESCRIPTION OF THE EMBODIMENTS

The present disclosure is directed to intermittent urinary catheters having proximal insertion tips, wherein the urinary catheters include at least one uniquely shaped eyelet positioned relatively close to the proximal terminal end of the catheter shaft.

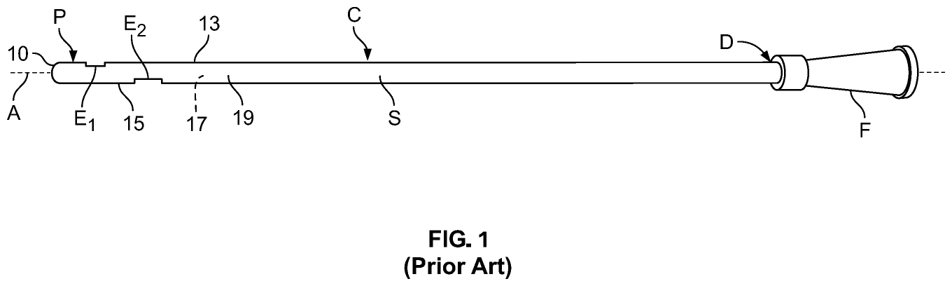

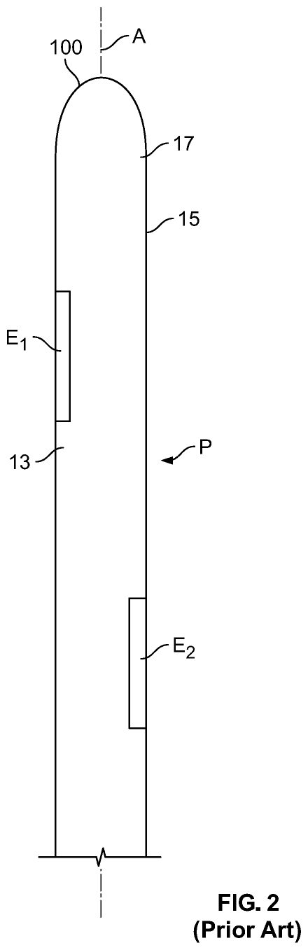

Turning now to the figures, a number of embodiments of urinary catheters of the present disclosure are shown, which may be intermittent urinary catheters. The figures illustrate the proximal insertion end portion of the catheters. Similar to FIG. 1, the urinary catheter may include a distal end portion (not shown). In the embodiments of the catheters disclosed herein, the distal end portion of the catheter shaft includes a drainage opening and, optionally, may include a drainage member (not shown) or be connected to a collection bag (not shown). Furthermore, the urinary catheter includes a drainage lumen that is in communication with the distal end drainage opening of the catheter shaft.

The proximal insertion end portion includes a terminal proximal end. In the embodiments disclosed herein, the terminal proximal end may be a closed end or an open end. When the terminal end is an open end, the terminal end includes an opening in communication with drainage lumen.

The proximal insertion end portion may include at least one eyelet. In some embodiments, the proximal insertion end portion may include at least two eyelets. The eyelet or eyelets may be arranged in the E1 and E2 position, as on the prior art catheter, with one on each of surfaces 13 and 15 of the urinary catheter. Additionally, when two or more eyelets are present, they may be alternatively arranged, such as two eyelets on surface 13 or surface 15 or two on one of surface 13 and 15 and one on one of surface 13 and 15.

The eyelet(s) extend through the proximal insertion end portion and are in communication with the drainage lumen. The urinary catheter shaft includes a sidewall having an outer surface and an inner surface. The eyelet(s) extend through the sidewall from the outer surface to the inner surface. The outer surface and the surfaces defining the eyelet may have a hydrophilic coating disposed thereon.

The proximal most eyelet may be located between about 2 mm and about 30 mm, preferably between about 8 mm and about 24 mm, or more preferably between about 5 mm and about 15 mm from the terminal proximal end 10 of the catheter.

There are a number of different shapes disclosed in the embodiments herein. In particular embodiments with more than one eyelet, each of the eyelets may vary in size and shape. In another alternatives, all of the eyelets are the same shape. In yet other alternatives, the eyelets may have a combination of shapes in the same embodiment.

The eyelets function to drain fluid that is external of the catheter to the inner lumen of the catheter. The eyelets disclosed herein each include an opening(s) or passageway(s) that extends through and is bounded by the catheter wall The cross-sectional area of each eyelet may vary depending on the embodiment. Eyelets disclosed herein may range from about 1.4 mm² to about 6.0 mm² or from about 1.6 mm² to 5.6 mm² in total open cross-sectional area.

When created, an eyelet may vary slightly in size, primarily due to finishing techniques (such as rounding of the edges in order to make the outside surface more comfortable for a user). The cross-sectional area of the drainage hole opening can be taken at any parallel plane to the center axis of the catheter.

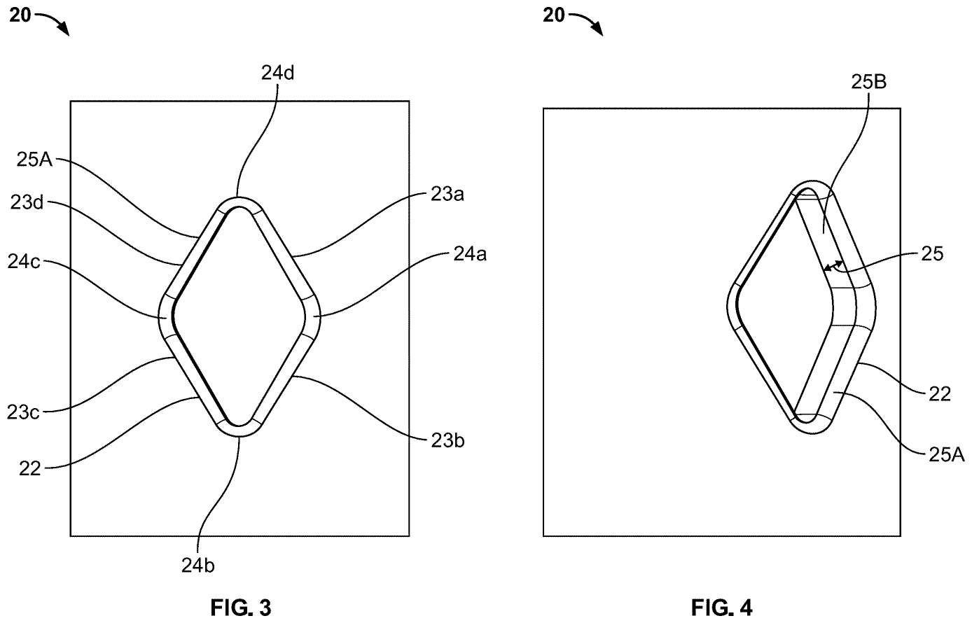

A number of different eyelet shapes are disclosed in FIGS. 3-12. These are non-limiting and other arrangements are within the scope of this disclosure. Although in the embodiments there is only one eyelet shown, it should be understood that a second eyelet of the same shape may also be present on the catheter in any given embodiment. Additional eyelets of the same shape may also be variously located. Additionally, each unique eyelet shape may be accompanied by a traditional slot or other shaped eyelet or eyelets on the proximal insertion end of the catheter.

FIGS. 3 and 4 show a possible embodiment of a portion of a proximal end of a catheter 20 with a catheter eyelet 22 in a diamond shape. A diamond-shaped eyelet maintains the current drainage rate of a typical oval/slot shaped eyelet but the ends of the eyelet gradually taper off to guide the tissue out gently rather than the more abrupt semi-circular shape.

The eyelet 22 has 4 straight portions 23a, 23b, 23c, and 23d for each of the four sides of the diamond. The eyelet 22 also has four rounded edges 24a, 24b, 24c, and 24d at the “points” of the diamond. The eyelet 22 can be on at least one of the top surface 13 and bottom surface 15 of the catheter, and may be located on each surface. Each catheter may have a plurality of catheter eyelets 22 arranged in different patterns. The catheter eyelets 22 may have a cross-sectional area of from about 1.0 mm² to about 5 mm² or from about 1.2 mm² to about 4.4mm². Each straight portion may be from about 0.4 mm to about 2.0 mm or about 0.5 mm to about 1.8 mm. Each of the portions 23 may be approximately the same length, but in other embodiments the lengths may vary with at least two different lengths present in the portions 23 of the eyelet 22. The catheter eyelet may have a depth 25 of about 0.30 mm to about 1.0 mm, or about 0.45 to about 0.8 mm, shown in FIG. 4. This depth may be consistent for the perimeter of the eyelet or vary at different points. The rounded edges may include a radius of curvature of from about 0.05 mm to about 0.5 mm. Additionally, the transition 25A between the outers surface of the catheter and the wall 25B of the eyelet may be a smooth or rounded transition that does not include any edges.

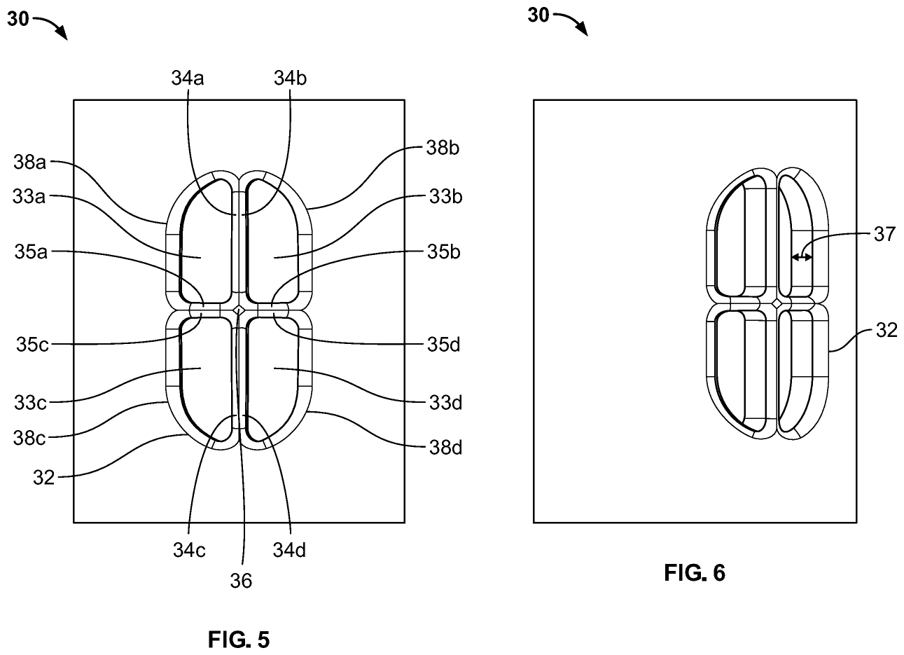

FIGS. 5 and 6 show a possible embodiment of a portion 30 of a proximal end of a catheter tube with eyelet 32. Eyelet 32 includes a shape or boundary with internal dividing portions making four hole portions 33a, 33b, 33c, and 33d that form a grille design. The overall shape is generally that of a slot or oval. The classic slot shaped outline with the addition of the grille design inside limits the ability of tissue to be drawn into the eyelet during insertion and removal, while still keeping the function and aesthetic of the slot eyelet design.

Each portion 33 includes three sides. All of the portions 33 intersect at point 36 at the center of the eyelet 32. Two of the three sides of each portion are adjacent to a neighboring portion. These two sides 34 and 35 are relatively straight. The other side 38 of each portion is rounded. Side 34a of portion 33a is adjacent to side 34b of portion 33b and side 35a is adjacent to side 35c of portion 33c. Side 34c of portion 33c is adjacent to side 34d of portion 33d. Side 35d of portion 33d is adjacent to side 35b of portion 33b. Each portion 33 may have a cross-sectional area from about 0.4mm² to about 1.4 mm² with the total cross-sectional area of the four portions, or eyelet 32 being from about 1.4 mm² to about 6.0 mm² or from about 1.6 mm² to 5.6mm². The four portions are aligned, as noted above, with adjacent sides, in order to form a clover leaf shape or an oval/slot with a cross through it. The eyelet has a depth 37 from 0.35 mm to about 1.0 mm or about 0.45 mm to about 0.8 mm which may be consistent through the eyelet or vary. Similar to as described above, the transitions between the outer surface of the catheter and the wall of the eyelet may be a smooth or rounded transition.

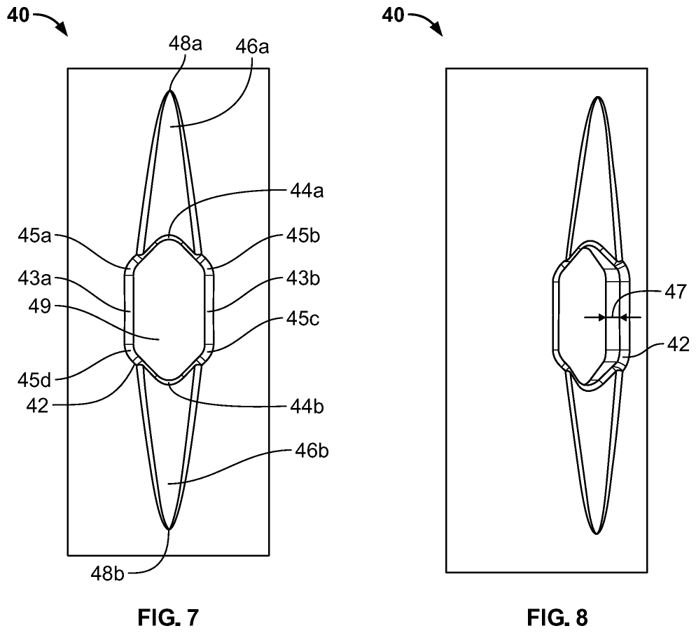

FIGS. 7-9 show a possible embodiment of a portion 40 of a proximal end of a catheter tube with eyelet 42. The eyelet 42 includes a shaped open middle portion 49 and two groove portions 46a, 46b on the top and bottom of the inner hole portion 49. Although two grooves are shown in the eyelet, the eyelets may have one or more grooves. An eyelet with the addition of an impression or groove, running in line with the direction of use aids in guiding tissue into and out of the eyelet in a gentler fashion. Furthermore, any of the eyelets disclosed herein may include one or more grooves extending proximally or distally from the eyelet. For example, grooves may extend proximally and/or distally from the eyelet shown in FIGS. 3 and 4.

The inner hole portion 49 may be of any desired shape, including a hexagon as shown in FIGS. 7-9. The hexagon can include six sides of equal length or sides or varying length, as shown in FIGS. 7 and 8. In an exemplary embodiment, open middle portion 49 is in the shape of a hexagon with two opposing sides 43a and 43b which are longer in length than the remaining sides. The open middle portion 49 of eyelet 42 also includes two sets of sides on either end of opposing sides 43a and 43b, sides 45a, 45b, 45c, and 45d. The edges of the hexagon are rounded and the top and bottom rounded edges are noted as 44a and 44b. The inner hexagon shape of the eyelet has a depth 47 from about 0.25 to about 1.0 mm or about 0.3 mm to about 0.8 mm which may be consistent or vary throughout the hexagon shape. The cross-sectional area of the inner hexagon may be from about 2.2 mm² to about 10 mm² or about 2.3 mm² to about 8.5mm². The length of the eyelet, including the groove portions, from the top 56a of groove portion 55a to the bottom 56b of groove portion 55b is from about 10 mm to about 30 mm.

Each of the top and bottom rounded edges 44a and 44b may include a groove portion extending from it. In an exemplary embodiment each of the top and bottom rounded edges has a groove portion 46a, 46b extending from it. The groove may vary in depth with a deeper portion near the inner shape at rounded edges 44a and 44b and a shallower portion near the tips 48a and 48b of the groove. In some embodiments, the deepest point of the groove portion may be near the open middle hole portion, and in other embodiments, the deepest point may be at another location along the groove portion. The depth of the groove may vary from about 0.05 mm to about 0.4 mm, with the deepest portion being from about 0.15 to about 0.4 mm and the shallowest portion being from about 0.05 mm to about 0.1 mm. The groove may be in a triangular or split cone shape. The tips 48a and 48b may be rounded. The length of each groove portion from the rounded edges 44a, 44b to the rounded tips 48a, 48b may be from about 2.5 mm to about 15 mm.

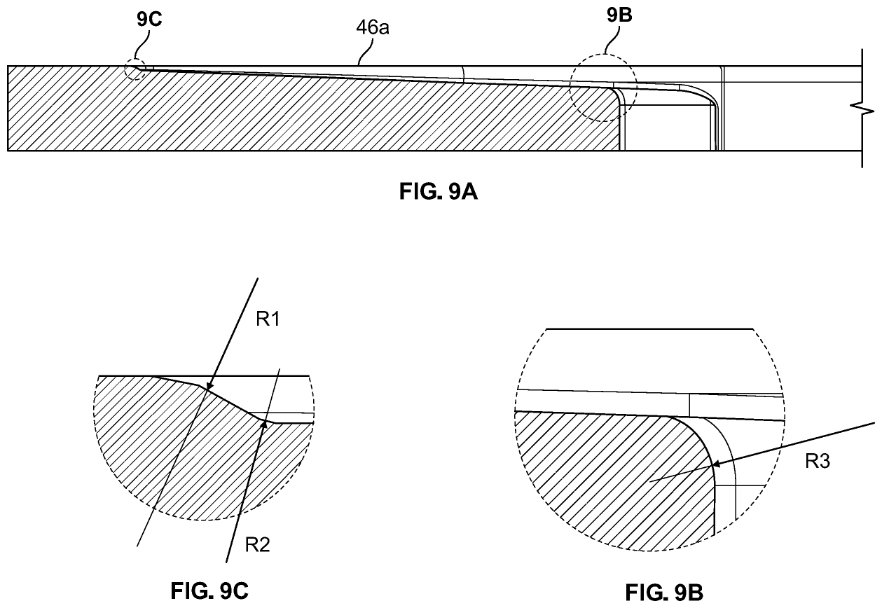

FIG. 9A includes a cross section of the catheter at the eyelet 42 of FIGS. 7 and 8. FIGS. 9B and 9C are expanded portions of the cross section of 9A. The radius of curvature for different points on the eyelet 42 are shown in FIGS. 9B and 9C, labeled R1, R2 and R3. FIG. 9B shows a radius R3 of 0.15 mm. FIG. 9C shows a radius R1 of 0.15 mm and a radius R2 of 0.05 mm.

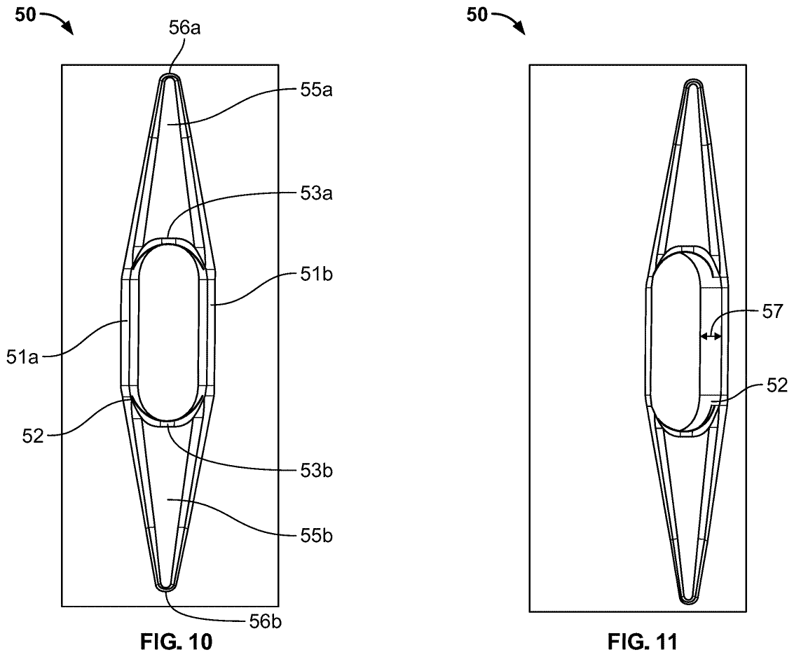

FIGS. 10-12 show a possible embodiment of a portion 50 of a proximal end of a catheter tube with eyelet 52. The eyelet 52 includes an open middle portion 59 and two groove portions 55a, 55b on the top and bottom. The open middle portion may be of any desired shape, including a slot or elongated oval as shown in FIGS. 10-12. The slot can include two longer straight sides 51a and 51b and two rounded top and bottom sides 53a and 53b. The inner slot shape of the open middle portion of the eyelet has a depth 57 from about 0.45 mm to about 0.8 mm. The cross-sectional area of the inner slot may be from about 1.1 mm² to about 3.9mm². The length of the eyelet, including the groove portions, from the top or tip 56a of groove portion 55a to the bottom or tip 56b of groove portion 55b is from about 10 mm to about 30 mm.

Each of the top and bottom rounded sides 53a and 53b may include a groove portion extending from it. In an exemplary embodiment each of the top and bottom rounded edges has a groove portion 55a, 55b extending from it. The groove may vary in depth with a deeper portion near the inner shape at rounded sides 53a and 53b and a shallower portion near the tips 56a and 56b of the groove. The depth of the groove may vary from about 0.05 mm to about 0.4 mm, with the deepest portion being from about 0.15 mm to about 0.4 mm and the shallowest portion being from about 0.05 mm to about 0.1 mm. The groove may be in a triangular or split cone shape. The tips 56a and 56b may be rounded. The length of each groove portion from the rounded sides 53a, 53b to the rounded tips 56 a, 56 b may be from about 2.5 mm to about 15 mm.

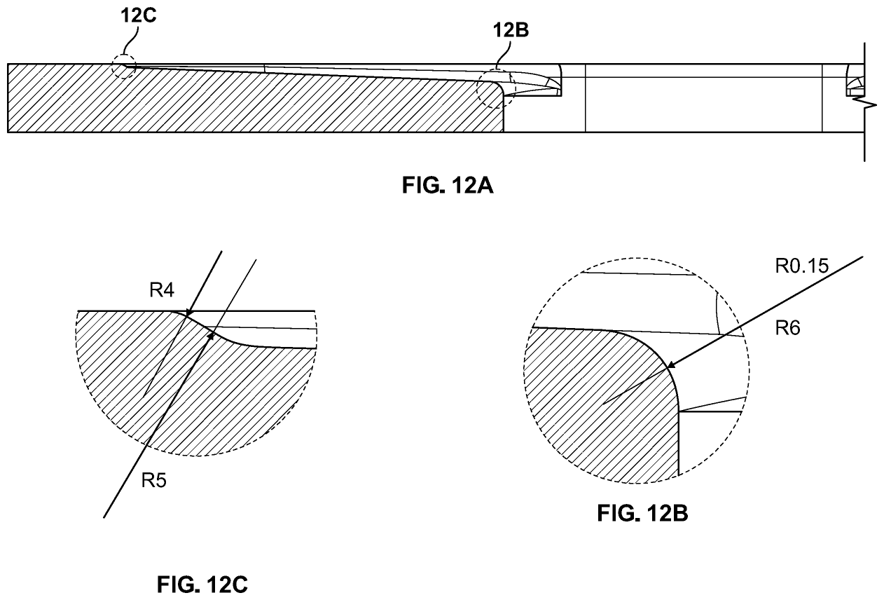

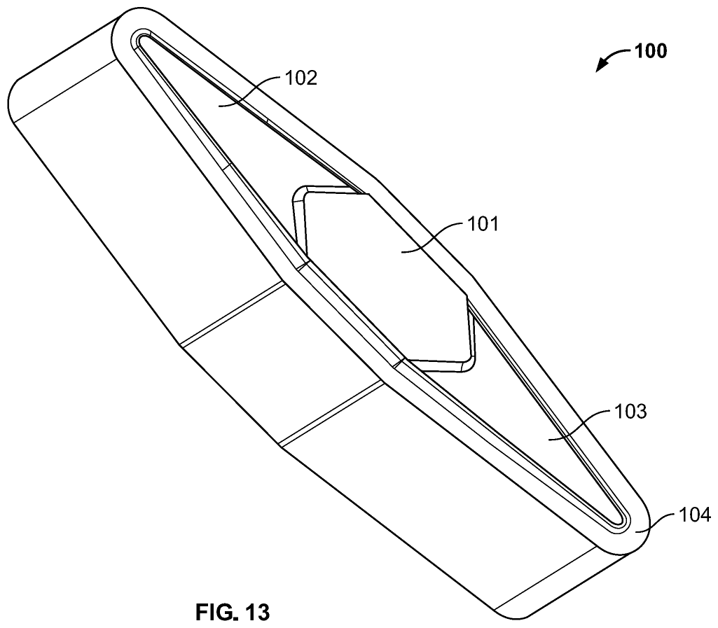

FIG. 12A includes a cross section of the catheter at the eyelet. FIGS. 12B and 12C are expanded portions of the cross section of 12A. FIGS. 12B and 12C are expanded portions of the cross section of 9A. The radius of curvature for different points on the eyelet 52 of FIGS. 10 and 11 are shown in FIGS. 12B and 12C, labelled R4, R5, and R6. FIG. 12B shows a radius R6 of 0.15 mm. FIG. 12C shows two different radiuses, R4 and R5, at two points, each one 0.10 mm.

The eyelets of the embodiments disclosed herein may be formed in any suitable manner, including but not limited to, mechanical punching, thermal forming, ultrasonic cutting, laser cutting, molded during a molding process, etc.

The eyelet types with groove portions (FIGS. 7-12) may be formed by a stamping and/or punching process. The process includes the use of at least two different stamps/dies and two different stamping actions. Two different example sets of dies are shown, the first in FIGS. 13-16 and the second in FIGS. 18-21. The difference between the two dies resides largely in the interior shape. While a hexagon and a slot are shown as possible interior shapes, any desired shape can be used as the interior shape.

A first die is applied to a catheter surface so that indentations are formed with a middle shape and two groove portions on the opposing sides of the middle shape. Afterward a second die is applied to the embossed or indented catheter surface that is formed after application of the first die, resulting in a middle open portion of the eyelet.

The first and/or second die may be heated or have other forms of energy applied thereto. Heat or other forms of energy may be added to the die before and/or during application of the first and/or second die. Additional forms of energy may include ultrasonic, radio frequency of laser energy, etc. In some alternatives, the temperature of the die and/or energy level is such that the pressure of the die against the catheter surface in combination with the heat/energy results in the desired form and/or cutout on the catheter surface.

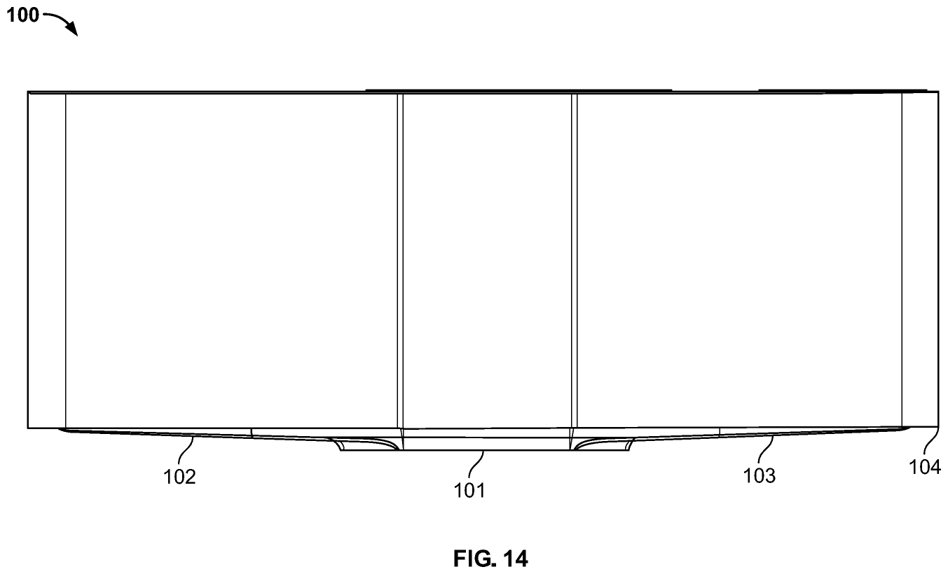

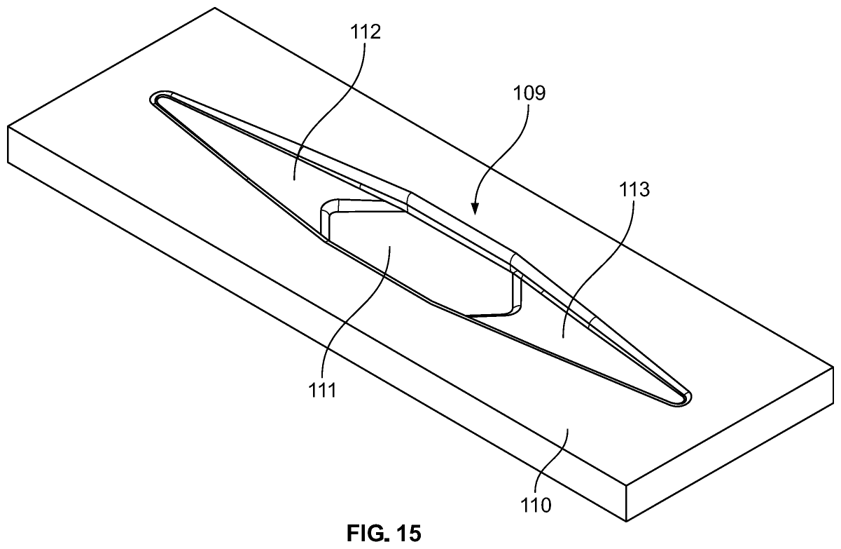

FIGS. 13-16 illustrate a first set of dies for forming the eyelet of FIGS. 7-9. The bottom of die 100, as shown in FIG. 13, includes a bottom surface 104 with a hexagon protruding portion 101 and protruding portions 102 and 103 that have the shape of a triangle with a concaved bottom, which corresponds to the grooved portion of an eyelet. Protruding portions 102 and 103 are located on opposing sides (top and bottom) of the hexagon protruding portion 101. FIG. 14 illustrates a side view of the die 100, clearly showing the protruding portions 101, 102, and 103, protruding from the bottom of the die. Portion 101 extends further from the bottom surface 104 of the die than portions 102 and 103. FIG. 15 shows a flattened portion of catheter 100 in which the first die 100 has been applied to, resulting in an indentation 109. Corresponding indentation portions 111, 112, and 113 are embossed into the catheter surface. Indentation portion 111 corresponds to the hexagon protruding portion 101. Each of the indentation portions 112 and 113 correspond to a protruding portion 102 and 103.

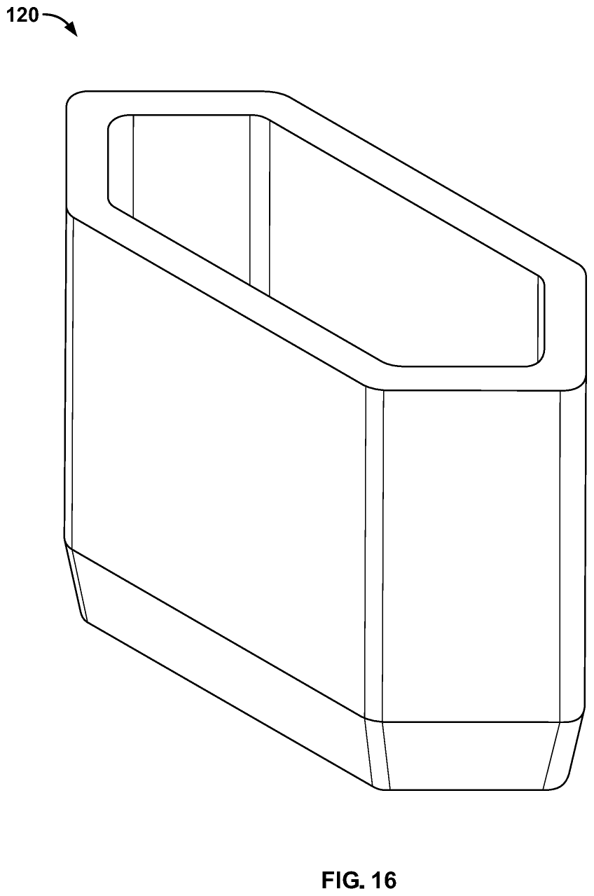

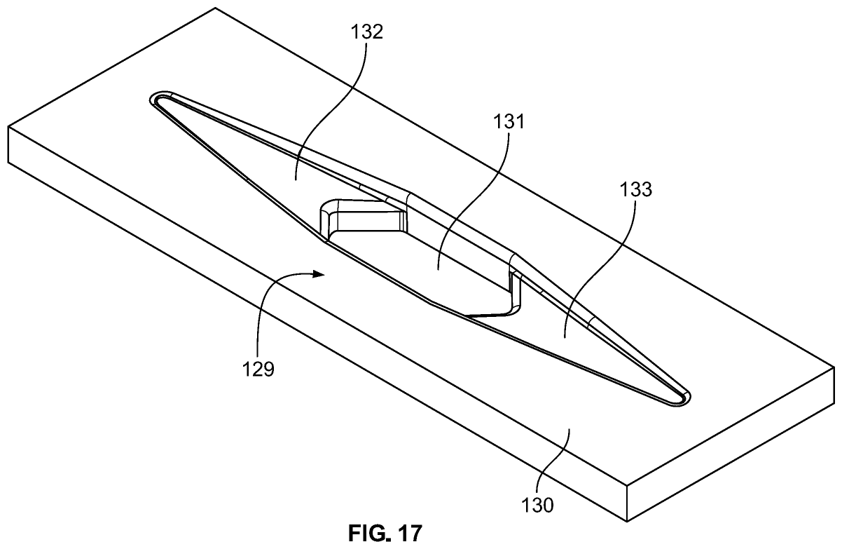

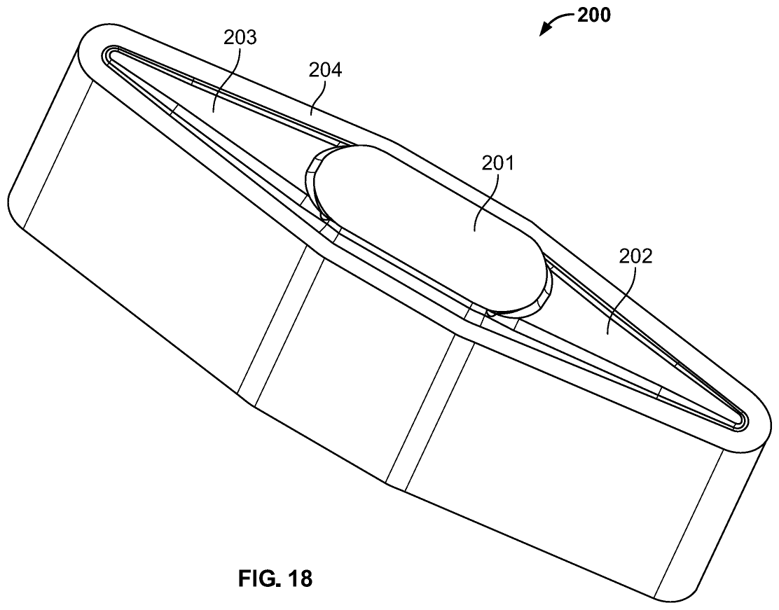

A second die 120 is shown in FIG. 16. This second die is then applied to the flattened idented surface or catheter 110 of FIG. 15. The second die has a stamping surface in the shape of the interior portion of the eyelet. FIG. 17 illustrates the resulting eyelet 129 on a flattened surface 130 of a catheter. Eyelet 129 includes two groove portions 132 and 133, which are unchanged from the application of the second die 120, and open hole portion 131, which corresponds to the hexagon portion 101. The second die removes the indentation portion 111 to form the hole 131.

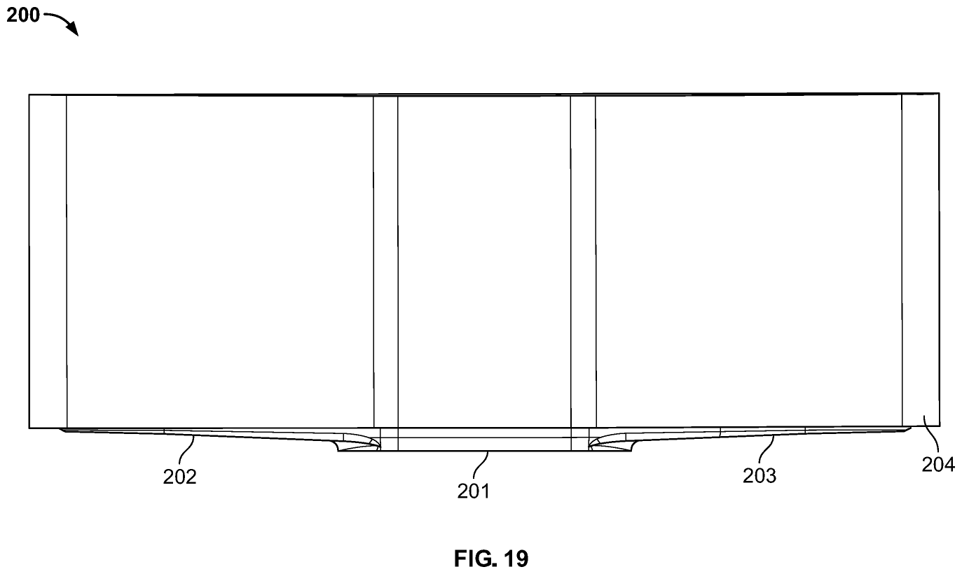

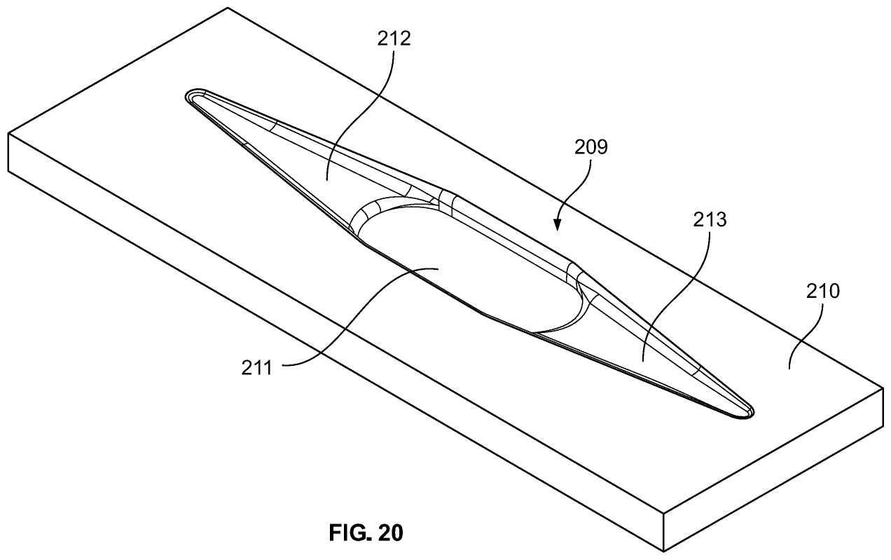

FIGS. 18-22 illustrate a second set of dies for forming the eyelet of FIGS. 10-12. The bottom of die 200, as shown in FIG. 18, includes a bottom surface 204 with an oval shaped protruding portion 201 and portions 202 and 203 that have the shape of a triangle with a concaved bottom, which corresponds to the grooved portion of an eyelet. Protruding portions 202 and 203 are located on opposing sides of the oval protruding portion 201. FIG. 19 illustrates a side view of the die 200, clearly showing the protruding portions 201, 202, and 203, protruding from the bottom of the die. Portion 201 extends further from the bottom surface 204 of the die than portions 202 and 203. FIG. 20 shows a flattened portion of the surface of catheter 210 in which the first die 200 has been applied to, resulting in an indentation 209. Corresponding indentation portions 211, 212, and 213 are embossed into the catheter surface. Indentation protruding portion 211 corresponds to the oval shaped protruding portion 201. Each of the indentation portions 212 and 213 correspond to protruding portions 202 and 203.

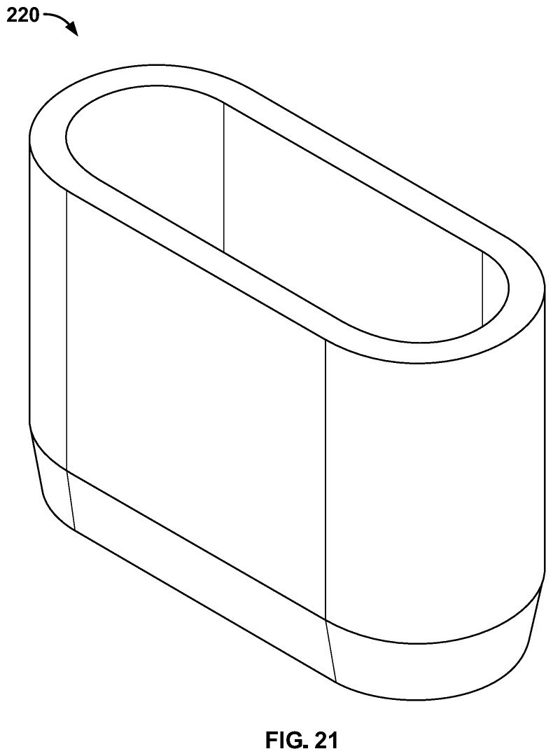

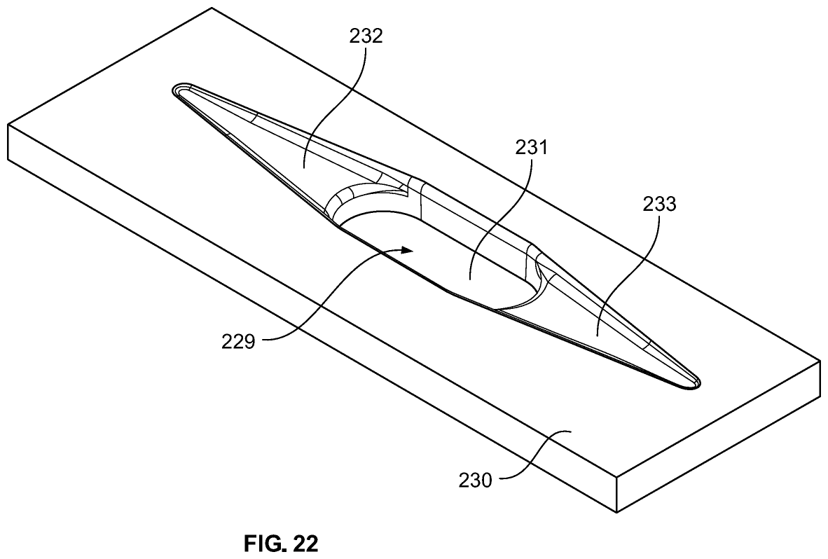

A second die 220 is shown in FIG. 21. The second die has a stamping surface in the shape of the interior portion of the eyelet. This second die is then applied to the flattened idented surface of catheter 210 of FIG. 20. FIG. 22 illustrates the resulting eyelet 229 on a flattened surface 230 of a catheter. Eyelet 229 includes two groove portions 232 and 233, which are unchanged from the application of the second die 220, and hole 231, which corresponds to the oval interior portion. The second die removed the indentation portion 111 to form the hole 231.

The examples described above are examples of catheters with eyelets and methods of forming these eyelets. Different combinations of these eyelets may be used or arranged.

It should be understood that various changes and modifications to the presently preferred embodiments described herein will be apparent to those skilled in the art. Such changes and modifications can be made without departing from the spirit and scope of the invention disclosed herein.

What is claimed is:

1. An intermittent urinary catheter, comprising:
a catheter shaft having a proximal insertion end portion for advancement through a urethra into a bladder and a distal drainage end portion having a drainage opening, the catheter shaft having a drainage lumen in communication with the drainage opening; and
at least one eyelet on the catheter shaft at the proximal insertion end portion;
wherein the at least one eyelet is diamond shaped.

2. The intermittent urinary catheter of claim 1, wherein the eyelet has rounded edges.

3. The intermittent urinary catheter of claim 1, wherein the catheter includes at least two eyelets.

4. The intermittent urinary catheter of claim 1, wherein each side of the at least one eyelet that is diamond shaped is about the same length.

5. The intermittent urinary catheter of claim 4, wherein a length of each side is about 0.4 mm to about 2.0 mm.

6. The intermittent urinary catheter of claim 1, preceding claims, wherein a cross sectional area of the at least one eyelet is from about 1.0 mm² to about 5mm².

7. The intermittent urinary catheter of claim 1, wherein a depth of the at least one eyelet is at least 0.3 mm to about 1.0 mm.

8. An intermittent urinary catheter, comprising:
a catheter shaft having a proximal insertion end portion for advancement through a urethra into a bladder and a distal drainage end portion having a drainage opening, the catheter shaft having a drainage lumen in communication with the drainage opening; and
at least one eyelet on the catheter shaft at the proximal insertion end portion;
wherein the at least one eyelet includes an outer shape divided into a number of adjoining portions.

9. The intermittent urinary catheter of claim 8, wherein the outer shape is a slot.

10. The intermittent urinary catheter of claim 8, wherein the number of adjoining portions is four.

11. The intermittent urinary catheter of claim 8, wherein the number of adjoining portions are about equal in size.

12. The intermittent urinary catheter of claim 8, wherein the eyelet has rounded edges.

13. The intermittent urinary catheter of claim 8, wherein the catheter includes at least two eyelets.

14. The intermittent urinary catheter of claim 8, wherein a cross-sectional area of the at least one eyelet is from about 1.4 mm² to about 6.0mm².

15. The intermittent urinary catheter of claim 8, wherein a depth of the at least one eyelet is at least 0.35 mm to about 1 mm.

16. An intermittent urinary catheter, comprising:
a catheter shaft having a proximal insertion end portion for advancement through a urethra into a bladder and a distal drainage end portion having a drainage opening, the catheter shaft having a drainage lumen in communication with the drainage opening; and
at least one eyelet on the catheter shaft at the proximal insertion end portion;
wherein the at least one eyelet includes an open middle portion and at least one outer groove portion.

17. The intermittent urinary catheter of claim 16, wherein the open middle portion is in a shape of a hexagon.

18. The intermittent urinary catheter of claim 17, wherein two opposing sides of the hexagon are longer than any remaining sides.

19. The intermittent urinary catheter of claim 16, wherein the open middle portion is a slot.

20. The intermittent urinary catheter of claim 16, wherein the eyelet includes at least two outer groove portions.

21.-33. (canceled)
